(19) United States
(12) Patent Application Publication
(10) Pub. No.: US 2011/0247404 A1
(43) Pub. Date: Oct. 13, 2011

(54) IDENTIFYING AND QUANTIFYING BIOMARKERS ASSOCIATED WITH PREECLAMPSIA

(75) Inventors: Steven W Graves, Highland, UT (US); Michael Sean Esplin, Salt Lake City, UT (US)

(73) Assignees: UNIVERSITY OF UTAH RESEARCH FOUNDATION, Salt Lake City, UT (US); IHC HEALTH SERVICES, INC., Salt Lake City, UT (US); BRIGHAM YOUNG UNIVERSITY, Provo, UT (US)

(21) Appl. No.: 13/000,480

(22) PCT Filed: Jun. 24, 2009
(86) PCT No.: PCT/US2009/048493

§ 371 Date: Jun. 27, 2011

Related U.S. Application Data
(60) Provisional application No. 61/075,361, filed on Jun. 25, 2008.

(51) Int. Cl. G01N 30/02; C07K 2/00

(52) U.S. Cl. 73/61.55; 530/300

(57) ABSTRACT

Described herein are methods for testing pregnant subjects for preeclampsia by detecting and quantifying at least one biomarker associated with preeclampsia in a biological sample from the subject.

### CROSS REFERENCE TO RELATED APPLICATIONS

This application claims priority upon U.S. provisional application Ser. No. 61/075,361, filed Jun. 25, 2008. This application is hereby incorporated by reference in its entirety.

### ACKNOWLEDGEMENTS

The research leading to this invention was funded in part by the National Institutes of Health, Grant Nos. R21HD047319 and U01HD050080. The U.S. Government may have certain rights in this invention.

### BACKGROUND

Preeclampsia (PE) is pregnancy induced hypertension in which protein is often observed in a subject's urine. This condition plagues pregnant mothers and their unborn children both domestically and abroad. For example, in the United States alone, PE affects approximately 3-5% of all pregnant women. More importantly, PE is the leading cause of perinatal maternal death in the United States and kills approximately 40,000 women worldwide each year.

PE is currently defined by an elevation in blood pressure (>140/90 mm Hg) and protein in the urine (>300 mg/24 hr) occurring in the second half of pregnancy in women without a history of high blood pressure, kidney disease, diabetes, or other significant disease. PE may also include a myriad of other abnormalities. Although no precise way to diagnose this condition exists, the possibility of PE is always considered for women displaying those particular symptoms beyond 20 weeks gestation. PE has proved particularly difficult to diagnose because its symptoms mimic many other diseases. If left undiagnosed or diagnosed too late, preeclampsia may progress to fulminant preeclampsia marked by headaches, visual disturbances, epigastric pain, and further to eclampsia.

Further adding to the complexity of diagnosis, some women exhibit preexisting hypertension, and preexisting hypertension is often difficult to discern from the onset of PE. To date, there is no effective way to diagnose this potentially fatal condition. Therefore, an important unmet need is to formulate a testing procedure for the early detection of mothers that will likely experience preeclampsia.

### SUMMARY

Described herein are methods for testing pregnant subjects for preeclampsia, which includes detecting and quantifying one or more biomarkers associated with preeclampsia in a biological sample from the subject. The biomarkers useful in predicting preeclampsia are also described in detail. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a biomarker” includes mixtures of two or more such biomarkers, and the like.

“Optional” or “optionally” means that the subsequently described event or circumstance can or cannot occur, and that the description includes instances where the event or circumstance occurs and instances where it does not.

As used herein, “subject” refers to a pregnant woman at risk of developing preeclampsia and benefits from the methods described herein.

As used herein, the term “biomarker” may be used to refer to a naturally-occurring biological molecule present in pregnant women at varying concentrations useful in predicting the risk of preeclampsia. For example, the biomarker can be a peptide present in higher or lower amounts in a subject at risk of developing preeclampsia relative to the amount of the same biomarker in a subject who did not develop preeclampsia during pregnancy. The biomarker can include other molecules besides peptides including small molecules such as but not limited to biological amines and steroids.

As used herein, the term “peptide” may be used to refer to a natural or synthetic molecule comprising two or more amino acids linked by the carboxyl group of one amino acid to the alpha amino group of another. A peptide of the present invention is not limited by length, and thus “peptide” can include polypeptides and proteins.

As used herein, the term “isolated,” with respect to peptides, refers to material that has been removed from its original environment, if the material is naturally occurring. For example, a naturally-occurring peptide present in a living animal is not isolated, but the same peptide, which is separated from some or all of the coexisting materials in the natural system, is isolated. Such isolated peptide could be part of a composition and still be isolated in that the composition is not part of its natural environment. An “isolated” peptide also includes material that is synthesized or produced by recombinant DNA technology.

As used herein, the term “detect” refers to the quantitative measurement of undetectable, low, normal, or high serum concentrations of one or more biomarkers such as, for example, peptides and other biological molecules.

As used herein, the terms “quantify” and “quantification” may be used interchangeably, and refer to a process of determining the quantity or abundance of a substance in a sample (e.g., a biomarker), whether relative or absolute.

As used herein, the term “about” is used to provide flexibility to a numerical range endpoint by providing that a given value may be “a little above” or “a little below” the endpoint without affecting the desired result.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Described herein are methods for identifying pregnant subjects that are at risk for developing preeclampsia. Particular biomarkers have been identified that may be utilized to identify pregnant subjects during early to mid-pregnancy that may later develop preeclampsia. Such markers may allow the diagnostic distinction between preeclampsia and other conditions that exhibit similar symptoms. Early identification of subjects at greater risk for preeclampsia would be of considerable value, as such subjects could be more closely monitored.

Testing of pregnant subjects using the methods described herein may occur at any time during pregnancy when biomarkers indicative of preeclampsia are quantifiable in the subject. For example, in one aspect biomarkers may be tested at from about 12 weeks to about 14 weeks gestation. It should be noted that these ranges should not be seen as limiting, as such testing may be performed at any point during pregnancy. Rather these ranges are provided to demonstrate periods of the gestational cycle where such testing is most likely to occur in a majority of subjects.

Useful biomarkers in identifying subjects at risk for preeclampsia include various peptides and other biological molecules. Certain peptides and other biological molecules have been identified using the techniques and methods described herein that correlate with the incidence of preeclampsia. Quantification of one or more of these peptides and other biological molecules provides some indication of the risk of preeclampsia for the subject, and thus may provide opportunities for preventative treatments. It should be noted that any biomarker that is predictive of preeclampsia should be considered to be within the scope of the claims of the present invention. In one aspect, however, nonlimiting examples of biomarkers associated with preeclampsia may include biological molecules and peptides found to be statistically different (p≦0.0001) from control subjects (i.e., pregnant women that do not develop preeclampsia).

In one aspect, a method for testing a pregnant subject for preeclampsia may include detecting the difference in concentration or amount of one or more biomarkers associated with preeclampsia present in a biological sample compared to a control (i.e., the relative concentration or amount of the biomarker(s) in a pregnant woman that does not develop preeclampsia). In one aspect, proteomic systems and methods can be used to identify and quantify the biomarkers. For example, comparing multiple mass spectra from different biological samples, locating mass ions that are quantitatively different after using approaches to compensate for non-biological variability, isolating, and characterizing the biomarker of interest can be used herein. Such a method may include fractionating each of a plurality of biological samples to form a plurality of elutions, obtaining a plurality of mass spectra from each of the plurality of elutions at a plurality of elution times, and finding a molecular ion peak of interest that appears to be quantitatively different between biological samples. The method may additionally include identifying a mass spectrum reference peak corresponding to an endogenous reference molecule that is substantially consistent between biological samples, the endogenous reference molecule having an elution time and a mass to charge ratio that are substantially similar to the peak of interest, and compensating for non-biological variation for each biological sample across the plurality of elutions by normalizing the peak of interest to a mass spectrum peak of the endogenous reference molecule. The method may further include conducting collision-induced fragmentation studies that use each of a plurality of collision energies one run at a time and summing resulting pluralities of fragment ion mass spectra without averaging to form a single cumulative daughter fragment mass spectrum, and use the daughter fragment mass spectrum to establish amino acid sequence data which is then used in identifying a peptide corresponding to a peak of interest in the single aligned mass spectrum.

In another aspect, a biological sample containing the biomarker(s) of interest can be fractionated to form a plurality of elutions, obtaining a plurality of mass spectra from each of the plurality of elutions at a plurality of elution times, and identifying a mass spectrum alignment peak corresponding to an endogenous alignment molecule that elutes in each of the plurality of elutions. The method may further include aligning the pluralities of mass spectra from each elution by aligning the mass spectrum alignment peak from each of the plurality of elutions, summing the pluralities of aligned mass spectra to form a single aligned mass spectrum, and identifying a peptide corresponding to a peak of interest in the single aligned mass spectrum. Although various techniques are contemplated, in one aspect aligning the pluralities of mass spectra may further include visually aligning the pluralities of mass spectra. Additionally, fractionating each of the plurality of biological molecules present in a plurality of biological samples may be accomplished by numerous methods, for example by capillary liquid chromatography (cLC). Specific methods and parameters for detecting and quantifying the biomarkers described herein are provided in the Examples.

The proteomic approaches used to detect and quantify the biomarkers make use of molecules native to all sera that serve as internal controls that can be used to correct for differences in specimen loading, ionization efficiency and mass spectrometer sensitivity. Further to above discussion, a peak is chosen as a reference if it can be shown to be quantitatively similar between comparison groups, elutes from the column in the same elution window as the candidate biomarker, is similar in its mass to charge ratio to that of the candidate biomarker, and is sufficiently abundant that every specimen will have a quantity that is more than 3 times the level of noise. The reference peaks described here are for quantitative correction of peak height or area that is related to specimen processing, chromatographic loading, ionization efficiency or instrumental sensitivity fluctuations but not due to biologic differences in peak quantity. This reference is termed an internal quantitative control.

Furthermore, individual masses may be defined by elution time (retention time). However, elution time (retention time) can also be expressed as a function of internal time controls. This is determined by the relative position of the peak of interest between the time maker that precedes the biomarker and the time marker that follows the peak of interest. This determination is deemed an R_{f} value. R_{f} values are calculated as follows:

R_{f}=(elution time of biomarker−elution time of preceding time marker)/(elution time of following time marker−elution time of preceding time marker).

In one aspect, the abundance of a biomarker is measured following processing and separation as a function of a reference molecule also present in the biological sample that serves as an internal control. The term “abundance” as used herein represents the number of ions of a particular mass measured by the mass spectrometer in a given mass spectrum or the sum of the number of ions of a specific mass observed in several mass spectra representing the full elution interval. Normalization of biomarker abundance to this internal control reduces non-biological variation and improves the ability to utilize biomarkers in risk prediction. Stated another way, by choosing a molecule for a reference that is present in a biological sample in an abundance that is relatively constant from one subject to another, variability in the processing of biological samples can be corrected for, particularly when comparing runs conducted on different days that may be spread out over long periods of time. As such, the relative abundance of a biomarker may vary depending on the particular biomarker involved. A particular cutoff value may therefore be established for each biomarker/reference ratio such that ratios of the biomarker peak abundance to the reference peak abundance above or below a certain value may be predictive of a substantially increased risk of preeclampsia during pregnancy. In one aspect, the abundance of a biomarker can be a machine derived value. For example, the abundance of a given biomarker can be represented by the number of ions of a particular mass measured by a mass spectrometer in a given mass spectrum or the sum of the number ions of a specific mass observed in several mass spectra representing the full elution interval.

Any type of biological sample that may contain a biomarker of interest may be screened, including such non-limiting examples as serum, plasma, blood, urine, cerebrospinal fluid, amniotic fluid, synovial fluid, cervical vaginal fluid, lavage fluid, tissue, and combinations thereof.

Using the techniques described above, nine biomarkers have been identified as indicators for preeclampsia. Specific details regarding the identification and quantification of the biomarkers is provided in the Examples. The first biomarker (“biomarker 1”), which is a peptide, has a mass ion peak (m/z) at 718.8, a mean mass of 4305.943±0.020 Daltons, a mean elution time of 20.40±0.83 minutes, and a R_{f} value of 0.635±0.85.

The second biomarker (“biomarker 2”), which is a peptide, has a mass ion peak (m/z) at 719.2, a mean mass of 4313.199±0.118 Daltons, a mean elution time of 20.24±0.77 minutes, and a R_{f} value of 0.737±0.072.

The third biomarker (“biomarker 3”), which is a peptide, has a mass ion peak (m/z) at 734.8, a mean mass of 1647.506±0.022 Daltons, a mean elution time of 19.40±1.42 minutes, and a R_{f} value of 0.294±0.024.

The fourth biomarker (“biomarker 4”) has a mass ion peak (m/z) at 649.3, a mean mass of 648.322±0.037 Daltons, a mean elution time of 24.27±0.67 minutes, and a R_{f} value of 0.343±0.120.

The fifth biomarker (“biomarker 5”) has a mass ion peak (m/z) at 507.3, a mean mass of 506.306±0.011 Daltons, a mean elution time of 17.64±0.67 minutes, and a R_{f} value of 0.359±0.039.

The sixth biomarker (“biomarker 6”) has a mass ion peak (m/z) at 1026.4, a mean mass of 2051.289±0.070 Daltons, a mean elution time of 28.02±0.99 minutes, and a R_{f} value of 0.134±0.032.

The seventh biomarker (“biomarker 7”) has a mass ion peak (m/z) at 639.3, a mean mass of 638.385±0.007 Daltons, a mean elution time of 30.15±0.71 minutes, and a R_{f} value of 0.175±0.097.

The eighth biomarker (“biomarker 8”) has a mass ion peak (m/z) at 942.5, a mean mass of 941.447±0.079 Daltons, a mean elution time of 17.37±0.68 minutes, and a R_{f} value of 0.915±0.013.

The ninth biomarker (“biomarker 9”) has a mass ion peak (m/z) at 1238.5, a mean mass of 1237.499±0.036 Daltons, a mean elution time of 19.04±0.56 minutes, and a R_{f} value of 0.270±0.101.

Although biomarkers 1-9 are present in most pregnant women, many pregnant women that go on to experience preeclampsia had either higher or lower blood serum concentrations of one or more of these biological molecules during pregnancy as compared to women that had normal births. For example, biomarker 1 was more abundant in PE cases while biomarker 2 was more abundant in the controls. Thus a comparison of the abundance of one or more of these biomarkers in a biological sample from a subject against a known control concentration from subjects that did not experience preeclampsia, or against a known biomarker concentration from the subject being tested, may be predictive of such complications. Those subjects having a higher or lower abundance of one or more of these biomarkers may have an increased risk of preeclampsia, and can thus be identified early enough to allow appropriate treatment. The abundance of a particular biomarker in predicting preeclampsia is described in detail below.

In one aspect, to calculate biomarker abundance of preeclamptic subjects and control subjects, either ratios or log ratios can be used. For example, the log ratio of log 718.8/719.2 (abundance of biomarker 1/abundance of biomarker 2) yielded a mean control (subjects who did not develop preeclampsia) of −0.440±0.205 and a mean PE (subjects at risk for later preeclampsia) of −0.0788±0.255 (Table 4 in Examples). Referring to Table 4 in the Examples, either ratios or the log ratios of the other biomarkers were calculated. The ratio of 734.8/742.8 (abundance of biomarker 3/abundance of reference peak) yielded a mean control of 0.630±0.073 and a mean PE of 1.026±0.059. In addition the log ratio of 734.8/742.8 (abundance of biomarker 3/abundance of reference peak) yielded a mean control of −0.278±0.045 and a mean PE of −0.022±0.025. The log ratio of log 649.3/512.3 (abundance of biomarker 4/abundance of reference peak) yielded a mean control of −0.098±0.386 and a mean PE of +0.315±0.323.

The ratio of 1026.4/518.3 (abundance of biomarker 6/abundance of reference peak) yielded a mean control of 0.163±0.019 and a mean PE of 0.0847±0.008. The ratio of 639.3/582.3 (abundance of biomarker 7/abundance of reference peak) yielded a mean control of 3.99±0.88 and a mean PE of 0.731±0.105. The ratio of 942.5/559.3 (abundance of biomarker 8/abundance of reference peak) yielded a mean control of 0.510±0.141 and a mean PE of 0.277±0.027. The ratio of 1238.5/623.4 (abundance of biomarker 9/abundance of reference peak) yielded a mean control of 2.473±0.290 and a mean PE of 1.917±0.322. Stated another way, a potentially preeclamptic subject would most likely exhibit an increase in biomarker 1, a decrease in biomarker 2, an increase in biomarker 3, an increase in biomarker 4, and a decrease in biomarker 5, a decrease in biomarker 6, a decrease in biomarker 7, a decrease in biomarker 8, and a decrease in biomarker 9 when compared to a subject that does not experience PE.

In certain aspects, the ratios or log ratios calculated above may be used to statistically predict the risk of pregnant women developing preeclampsia. One common measure of the predictive power of a biomarker is its sensitivity and specificity. “Sensitivity” as used herein is a statistical term defined as the true positive rate (e.g., the percentage of pregnant women who later develop preeclampsia that are correctly identified by the biomarker). The term “specificity” as used herein is defined as the true negative rate (e.g., the percentage of pregnant women with uncomplicated pregnancies correctly identified). To use a biomarker as described herein for predicting preeclampsia, a numeric threshold is established. To establish a numeric threshold, the range of values for the specific biomarker are considered from lowest to highest and at each point the percent of subjects correctly identified as positive and at that same point the percent of controls incorrectly identified as positive. The range of values for the specific biomarker may be calculated by taking the actual quantative value from the lowest to highest for a specific data set. This is termed a receiver operator curve (ROC). In one aspect, the false positive rate can be limited to 20%, which is commonly considered the maximum value tolerated for a clinical test. The false positive rate (i.e., the percentage of women with uncomplicated pregnancies identified by the biomarker as at risk for developing preeclampsia) is calculated from the true negative rate subtracted from 100%. The threshold at a false positive rate of 20% or less, which is equivalent to a specificity of 80% or higher, determines the threshold used to determine whether someone is at risk or is not at risk.

Ratios and log ratios of the biomarkers were used to further determine specificity and sensitivity. Referring to Table 5 in the Examples, a threshold for each of the four log ratios was determined for the identification of subjects at risk of developing preeclampsia. The threshold for each was calculated such that there would be a specificity (a true negative rate) of 80% or more, which is the same as a false positive rate of no more than 20%. Using the mathematically determined thresholds, the four ratios independently provided sensitivity (true positive) and specificity (true negative) rates (Table 5). Referring to Table 5, the ratio of biomarker 1/biomarker 2 provided the greatest sensitivity (82%) and specificity (85%) with respect to predicting the development of preeclampsia. Thus, in this aspect, the identification and quantification of biomarker 1 and 2 is present in pregnant women is an accurate predictor of the likelihood of developing preeclampsia. Although the ratio of biomarker 1/biomarker 2 is useful, it is also contemplated that the combination of log ratios can be used to predict the risk of preeclampsia. For example, if the log 718.8/719.2 ratio (at a threshold of >−0.301) is combined with the ratio of log 649.3/512.3 at a threshold of >0.301, the sensitivity improves to 89.3% with a specificity of 85% (see Examples).

In one aspect, the weighted combination of the ratios for biomarker 3 (i.e. abundance of 734.8/abundance of 742.4), biomarker 6 (i.e. abundance of 1026.4/abundance of 518.3), biomarker 7 (i.e. abundance of 639.3/abundance of 582.3), and the ratio of 718.8/719.2 (i.e., ratio of biomarkers 1/2) can be used to improve sensitivity and specificity. In this aspect the weighted combinations are calculated as the following: [(−5×ratio 734/742)+(33×ratio 1026/518)+(2×ratio 639/582)+(−2×ratio 718/719)]=weighted value. If the weighted value >0.0, then this is indicative of an uncomplicated pregnancy thereafter. If the weighted value <0.0, then this is indicative of an increased risk of preeclampsia. As discussed in the examples, this method provided 96% sensitivity and 100% specificity.

In another aspect, the weighted combination of the ratios for biomarker 3 (i.e. abundance of 734.8/abundance of 742.4), biomarker 6 (i.e. abundance of 1026.4/abundance of 518.3), biomarker 7 (i.e. abundance of 639.3/abundance of 582.3), and biomarker 9 (i.e. abundance of 1238.5/abundance of 623.4) can be used to calculate sensitivity and specificity. In this aspect the weighted combinations are calculated as the following: [(−16×ratio 734/742)+(64×ratio 1026/518)+(3×ratio 639/582)+(1×ratio 1238/623)]=weighted value. If the weighted value >0.0, then this is indicative of an uncomplicated pregnancy thereafter. If the weighted value <0.0, then this is indicative of an increased risk of preeclampsia. As discussed in the examples, this method provided 96% sensitivity and 96% specificity.

Thus, the biomarkers identified herein are powerful tools in predicting the risk of preeclampsia.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Serum Collection

Studies involved 55 pregnant women having blood withdrawn between 12 and 14 weeks of pregnancy who were followed through the completion of their pregnancy. Twenty seven of these women had uncomplicated pregnancies with no evidence of preeclampsia (PE) including no increase in blood pressure or abnormal levels or protein in their urine. These constituted the control group. Twenty eight of these women developed later PE, each after 24 weeks of pregnancy. These women constituted cases of PE. The sera of these 55 women were studied using the proteomics approach.

### Acetonitrile Precipitation

Two volumes of HPLC grade acetonitrile (400 μL) were added to 200 μL of serum, vortexed vigorously for 5 sec and allowed to stand at room temperature for 30 min. Samples from (Serum collection) were then centrifuged for 10 min at 12,000 rpm in and IEC Micromax RF centrifuge (Thermo Fisher Scientific, Waltham, Mass.) at room temperature. An aliquot of supernatant was then transferred to a microcentrifuge tube containing 300 μL HPLC grade water. The sample was vortexed briefly to mix the solution which was then lyophilized to ˜200 μL in a Labconco CentriVap Concentrator (Labconco Corporation, Kansas City, Mo.). The volume of water added prior to lyophilization aids in the complete removal of acetonitrile from the solution. This is necessary because acetonitrile is incompatible with the assay used to determine protein concentration. Supernatant protein concentration were determined using a Bio-Rad microtiter plate protein assay performed according to manufacturer's instructions. An aliquot containing 4 μg of protein was transferred to a new microcentrifuge tube and lyophilized to near dryness. Samples were brought up to 20 μL with HPLC water and then acidified using 20 μL 88% formic acid.

Acetonitrile treated (post precipitation) serum samples (40 μL) were loaded into 250 μL conical polypropylene vials closed with polypropylene snap caps having septa (Dionex Corporation, Sunnyvale, Calif.), and placed into a FAMOS® autosampler 48 well plate kept at 4° C. The FAMOS® autosampler injected 5 μL of each serum sample onto a liquid chromatography guard column using HPLC water acidified with 0.1% formic acid at a flow rate of 40 μL/min. Salts and other impurities were washed off of the guard column with the acidified water. Because the FAMOS® autosampler draws up three times the volume of what is loaded onto the column, it was necessary to inject the samples by hand when sample volume was limited. This was accomplished by injecting 10 μL volume sample onto a blank loop upstream of the guard column and programming the FAMOS® autosampler to inject a 10 μL sample of HPLC water in place of the sample. The serum sample was loaded onto the guard column an desalted as if it had been loaded from the conical vials.

### Liquid Chromatography Separation for Mass Spec Analysis

Capillary liquid chromatography (cCL) was performed to fractionate the sample. Capillary LC uses a 1 mm (16.2 μL) microbore guard column (Upchurch Scientific, Oak Harbor, Wash.) and a 15 cm×250 μm i.d. capillary column assembled in-house. The guard column was dry-packed and the capillary column was slurry packed using POROS R1 reversed-phase media (Applied Biosystems, Framingham, Mass.). Column equilibration and chromatographic separation were performed using an aqueous phase (98% HPLC grade H₂O, 2% acetonitrile, 01.% formic acid) and an organic phase (2% HPLC H₂O, 98% acetonitrile, 0.1% formic acid). Separation was accomplished beginning with a 3 min column equilibration at 95% aqueous solution, followed by a 2.75%/min gradient increase to 60% organic phase, which was then increased at 7%/min to a concentration of 95% organic phase. The gradient was held at 95% organic phase for 7 min to elute the more hydrophobic components of the sample, and then the gradient was returned to 95% aqueous phase over 5 min and held at this concentration for 2 min to re-equilibrate the column. All separations were performed at a flow rate of 5 μL/min. Chromatography used an LC Packings Ultimate Capillary HPLC pump system, with FAMOS® autosampler (Dionex Corporation, Sunnyvale, Calif.), controlled by the Analyst QS® (Applied Biosystems, Foster City, Calif.).

### MS Analysis

MS calibrations were performed using an external control daily prior to running samples. If needed, settings were adjusted to optimize signal to noise ratio and to maximize sensitivity.

The cLC system was coupled directly to a mass spectrometer. Effluent from the capillary column was directed into a QSTAR Pulsar 1 quadrupole orthogonal time-of-flight mass spectrometer through an IonSpray source (Applied Biosystems). Data was collected for m/z 500 to 2500 beginning at 5 min and ending at 55 min. The delay in start time was programmed because, with a flow rate of 5 μL/min, it takes over 5 min for sample to get from the guard column to the mass spectrometer, and thus no useful data can be obtained before 5 min. Data collection, processing and preliminary formatting are accomplished using the Analyst QS® software package with BioAnalyst add-ons (Applied Biosystems).

Mass spectra were obtained every 1 sec throughout the entire cLC elution period for each specimen from 5 minutes to 55 minutes. The elution profile of the cLC fractionated protein depleted serum of each subject, reported as the total ion chromatogram, was inspected to insure that it was consistent with previously run human sera. Specimens having an overall abundance less than 50% of normal or greater than 200% normal or lacking the characteristic series of three broad ion intense regions were rerun or omitted if there was inadequate specimen to redo the analysis.

### Peak Alignment

Because samples run on different days and columns can vary in elution time, 10 endogenous molecular species of average abundance that elute at approximately 2 minute intervals throughout the useful chromatogram (useful chromatogram approximately 15 minutes to 35 minutes) were determined. Two-minute windows were established over the elution region of interest to allow file size to remain manageable. The Extract Ion Chromatogram (XIC) function of the MS computer is used to visualize the elution of the desired m/z ranges for each elution time marker. Each of the alignment peak's elution time is then determined for each specimen run and in turn used as the center of a 2 min window by means of the Set Selection function. This aligns all runs to the same midpoint for that window. Then the Show Spectra function can be used to create a single averaged mass spectrum from all the mass spectra.

### Data Analysis

Analyst®, the software program supporting the Q-Star (q-TOF) mass spectrometer, allows for compilation of 16 individual liquid chromatographic runs and the comparison of mass spectra within those runs at similar elution times. Ten two-minute windows were established as described above over the 20 minute period of useful elution to allow data file size to remain manageable. The two minute windows were aligned as is also described above. Of the 10 two minute elution intervals, the first to be analyzed was the second two-minute window, chosen because there were typically more peptide species present. Peptides were identified by the characteristic appearance of their multiply charged states which appear as a well defined cluster of peaks having a Gaussian shape with the individual peaks being separated by less than 1 mass/charge unit rather than a single peak or peaks separated by 1 mass/charge unit. Groups comprising 8 subjects from preeclamptic cases and 8 subjects from controls were color coded and overlaid. The data was then visually inspected and molecular species that seemed to be dominated by one color were recorded. The software used was limited to visualizing the mass spectra only 16 samples. For a sampling size larger than 16, multiple comparisons of data sets were made. For a compound to be considered further, the same apparent difference between the two groups was needed to be observed in at least two thirds of the data sets.

Molecules that appeared to be different between the two study groups were then individually inspected. These candidate species were all peptides. Prior to extracting quantitative data, the mass spectrum was examined to insure that the peptide peak had the same m/z and also represented the same charge state to further insure that the same peptide was being considered. Additionally, a second nearby peak, which did not demonstrate differences in abundance between the two groups, was selected as a reference. This peak was used to normalize the candidate peak of interest and correct for variability in specimen processing, specimen loading and ionization efficiencies.

The molecular species are then ‘extracted’ by the Analyst® software to determine the peak maxima of the individual molecular species in each individual run. This feature did not limit inspection of a specific m/z to a two minute elution window and consequently the peak used to align cLC elution time may be used additionally to insure the location in the elution profile was the same and hence insure that the same molecular species was selected each time.

The peak height for each molecular species was considered a reasonable estimate of its abundance. The abundance of each candidate compound was tabulated and the calculated value of each candidate species was ratioed to the nearby reference species. Because a single species was being considered, univariate statistical analysis was employed in evaluating possible differences in this peptide's abundance between the two groups.

### Endogenous Time Alignment Molecules

The mass and typical elution time of the reference peaks used for time alignment are summarized in Table 1.

TABLE 1
Mass and Elution Time of the Time Alignment Markers

| Mass of Endogenous Time Reference (daltons) | Mean Elution Time (min) |
|---|---|
| 1464.65 | 14.68 |
| 1439.52 | 17.01 |
| 2009.95 | 18.83 |
| 5062.28 | 21.34 |
| 546.31 | 23.54 |
| 545.33 | 26.12 |
| 1046.67 | 27.60 |
| 636.31 | 32.44 |
| 779.52 | 34.59 |
| 1619.07 | 36.88 |

Knowledge of the location of these endogenous molecular species present in all sera of pregnant women also allows them to be used for time markers for the alignment and localization of the PE biomarkers within capillary liquid chromatography elution profile.

### Biomarker Characteristics

After time alignment, biomarker candidates were identified visually in an initial process where multiple mass spectra were overlaid with cases and controls each assigned a color. Those peaks that appear to be predominantly one color were studied further. The individual spectra were then submitted to peak height determination by the computer equipped with Analyst® software (Applied Biosystems) which is the operating system for the QqTOF mass spectrometer (Applied Biosystems). The quantity of the biomarkers was then tabulated. In addition, a second peak that occurred in the same time window which was not quantitatively different between cases and controls was also selected. This represented a endogenous control to allow for reduction of non-biologic variability. This was accomplished by dividing the quantity of the candidate peak by the quantity of the endogenous control. The magnitude of the ratio for each specimen was recorded and statistical differences were sought using a Student's t test comparing cases and controls.

Nine species were sufficiently different (p≦0.0001) to suggest that they might allow for excellent separation of the two groups. The individual masses and elution time for the nine PE biomarkers are summarized in Table 2.

TABLE 2
Mass and Elution Time of the Biomarkers

| | Peak (m/z) | Mean Mass | Mean Elution Time |
|---|---|---|---|
| 1. | 718.8 | 4305.943 ± 0.020 | 20.40 ± 0.83 |
| 2. | 719.2 | 4313.199 ± 0.118 | 20.24 ± 0.77 |
| 3. | 734.8 | 1647.506 ± 0.022 | 19.40 ± 1.42 |
| 4. | 649.3 | 648.322 ± 0.037 | 24.27 ± 0.67 |
| 5. | 507.3 | 506.2 ± 0.011 | 17.64 ± 0.67 |
| 6. | 1026.4 | 2051.289 + 0.070 | 28.02 + 0.99 |
| 7. | 639.3 | 638.385 ± 0.007 | 30.15 ± 0.71 |
| 8. | 942.5 | 941.447 ± 0.079 | 17.37 ± 0.68 |
| 9. | 1238.5 | 1237.499 ± 0.036 | 19.04 ± 0.56 |

The elution time (retention time) was expressed as a function of the internal time controls. This was determined by the relative position of the peak of interest between the time marker that precedes the biomarker and the time marker that followed the peak of interest. This was calculated by the following formula:

R_{f}=(elution time of biomarker−elution time of preceding time marker)/(elution time of following time marker−elution time of preceding time marker)

The R_{f} values were more reliable than the actual elution times. Elution times may vary with new columns or with the altered performance of an existing column with fouling, but the R_{f} was not altered by these changes. The R_{f} values of the nine biomarkers are provided in Table 3.

TABLE 3
The R_{f} Values for the PE Biomarkers
Using the Internal Time Alignment Peaks.

| | Peak (m/z) | N | R_{f} Value Relative To Boundary Time Markers |
|---|---|---|---|
| 1. | 718.8 | 12 | 0.635 ± 0.85 |
| 2. | 719.2 | 12 | 0.737 ± 0.072 |
| 3. | 734.8 | 9 | 0.294 ± 0.024 |
| 4. | 649.3 | 10 | 0.343 ± 0.120 |
| 5. | 507.3 | 11 | 0.359 ± 0.039 |
| 6. | 1026.4 | 8 | 0.134 ± 0.032 |
| 7. | 639.3 | 8 | 0.175 ± 0.097 |
| 8. | 942.5 | 8 | 0.915 ± 0.013 |
| 9. | 1238.5 | 8 | 0.270 ± 0.101 |

### Reduction of Variability by Reference to an Endogenous Coeluting Control

One of the features of the current serum proteomic approach is the use of an endogenous molecule that was found in all species and was not different between cases and controls. Normalization of biomarker abundance to this internal control reduced non-biological variation and improved the ability to utilize biomarkers in risk prediction. Normalization involved mathematically dividing the abundance of the peak of interest by the reference peak. The abundances were machine derived values. The abundance of a given molecule represents the number of ions of a particular mass measured by the mass spectrometer in a given mass spectrum or the sum of the number ions of a specific mass observed in several mass spectra representing the full elution interval. Molecules typically require 1.0-1.5 min to move off the chromatographic column whereas mass spectra are acquired every 1 second during that elution interval.

The first two peaks with m/z 718.8 and 719.2 were both significantly different between cases and controls but the first was more abundant in PE cases and the second was more abundant in controls. These two peaks were referenced to each other, i.e. the abundance of the m/z 718.8 was divided by the abundance of the m/z 719.2. For the other three biomarkers, internal references were used. For the biomarker m/z 734.8, a coeluting reference peak at m/z 742.4 was used. For the biomarker m/z 649.3, a coeluting reference peak at m/z 512.3 was chosen. For the biomarker m/z 507.3, a coeluting reference at m/z 734.5 was chosen. For the biomarker m/z 1026.4, a coeluting reference at m/z 518.3 was chosen. For the biomarker m/z 639.3, a coeluting reference at m/z 582.3 was chosen. For the biomarker m/z 942.5, a coeluting reference at m/z 559.3 was chosen. For the biomarker m/z 1238.5, a coeluting reference at m/z 623.4 was chosen.

The mean value for either the ratios or log ratios or were calculated (Table 4):

TABLE 4
Biomarker Abundance (after Normalization) in Cases and Controls

| | Ratio | | Mean Control | Mean PE | P value |
|---|---|---|---|---|---|
| 1. | log | 718.8/719.2 | −0.440 ± 0.205 | −0.0788 ± 0.255 | 2 × 10⁻⁷ |
| 2. | | 734.8/742.3 | 0.630 ± 0.073 | 1.026 ± 0.059 | 0.00018 |
| 3. | log | 649.3/512.3 | −0.098 ± 0.386 | +0.315 ± 0.323 | 0.00003 |
| 4. | log | 507.3/734.5 | +0.400 ± 0.524 | −0.0944 ± 0.3962 | 0.0001 |
| 5. | | 1026.4/518.3 | 0.163 ± 0.019 | 0.0847 ± 0.008 | 0.00073 |
| 6. | | 639.3/582.3 | 3.99 ± 0.88 | 0.731 ± 0.105 | 0.0009 |
| 7. | | 942.5/559.3 | 0.164 ± 0.019 | 0.085 ± 0.008 | 0.00073 |
| 8. | | 1238.5/623.4 | 2.473 ± 0.290 | 1.917 ± 0.322 | 0.021 |

### Use of the Biomarkers to Predict Women at Risk of Developing Preeclampsia

As described above, one common measure of the predictive power of a biomarker was its sensitivity and specificity. A threshold for each of the four log ratios in Table 4 was determined in order to identify subjects at risk of developing PE. The threshold for each was calculated such that there would be a specificity (a true negative rate) of 80% or more. As stated, this is the same as a false positive rate of no more than 20%. Using these mathematically determined thresholds the four ratios independently provided the following sensitivity (true positive) and specificity (true negative) rates as summarized in Table 5.

TABLE 5
Sensitivity and Specificity of Each Biomarker (after Normalization)

| | Ratio | Threshold | Sensitivity | Specificity |
|---|---|---|---|---|
| 1. | log 718.8/719.2 | ≧−0.301 | 82% | 85% |
| 2. | log 734.8/742.4 | ≧−0.11 | 71% | 85% |
| 3. | log 649.3/512.3 | ≧0.253 | 67% | 80% |
| 4. | log 507.3/734.5 | ≦−0.125 | 48% | 85% |

The first two biomarkers when used in combination resulted in greater than 80% sensitivity and specificity for detecting the risk of preeclampsia. If the log 718.8/719.2 ratio (at a threshold of >−0.301) was combined with the ratio of log 649.3/512.3 at a threshold of >0.301, the sensitivity improved to 89.3% with a specificity of 85%. Combination of the log 718.8/719.2 ratio with log 734.8/742.4 at a threshold >−0.10 provided a sensitivity of 100% with a specificity of 74%.

Table 6 shows weighted combinations of various biomarkers used to identify and quantify subjects who are at risk for PE. For example, the combination of the ratios for biomarker 3 (abundance of 734.8/abundance of 742.4), biomarker 6 (abundance of 1026.4/abundance of 518.3), biomarker 7 (abundance of 639.3/abundance 582.3), and the ratio of 718.8/719.2 (i.e., ratio of biomarkers 1/2) were used to calculate sensitivity and specificity. The weighted combinations were calculated as follows: [(−5×ratio 734/742)+(33×ratio 1026/518)+(2×ratio 639/582)+(−2×ratio 718/719)]=weighted value. If the weighted value >0.0, then this was indicative of an uncomplicated pregnancy thereafter. If the weighted value <0.0, then this was indicative of preeclampsia in the pregnancy. This weighted combination provided 96% sensitivity and 100% specificity.

Combination the ratio for biomarker 3 (abundance of 734.8/abundance of 742.4), biomarker 6 (abundance of 1026.4/abundance of 518.3), biomarker 7 (abundance of 639.3/abundance of 582.3), and biomarker 9 (abundance of 1238.5/abundance of 623.4) can be used to calculate sensitivity and specificity. In this aspect the weighted combinations were calculated as the following: [(−16×ratio 734/742)+(64×ratio 1026/518)+(3×ratio 639/582)+(1×ratio 1238/623)]=weighted value. If the weighted value >0.0, then this was indicative of an uncomplicated pregnancy thereafter. If the weighted value <0.0, then this was indicative of preeclampsia in the pregnancy. This weighted combination provided 96% sensitivity and 96% specificity.

TABLE 6
Weighted Combinations of PE Biomarkers

| | | | | | Sensitivity | Specificity |
|---|---|---|---|---|---|---|
| mass | 734.8 | 1026.4 | 639.3 | 718/719 | 96% | 100% |
| Rel. | −5 | 33 | 2 | −2 | | |
| Weight | | | | | | |
| mass | 734.8 | 1026.4 | 639.3 | | 87% | 100% |
| Rel. | −3 | 17 | 1 | | | |
| Weight | | | | | | |
| mass | 734.8 | 942.5 | 1026.4 | 634.3R | 96% | 96% |
| Rel. | −4 | 2 | 18 | 3 | | |
| Weight | | | | | | |
| mass | 1238.5 | 734.8 | 1026.4 | 639.3 | 96% | 96% |
| Rel. | 1 | −16 | 64 | 3 | | |
| Weight | | | | | | |
| mass | 734.8 | 942.5 | 1026.4 | 634.3R | 96% | 96% |
| Rel. | −2 | 1 | 9 | 2 | | |
| Weight | | | | | | |
| mass | 734.8 | 1026.4 | 634.4R | | 91% | 96% |
| Rel. | −1 | 6 | 1 | | | |
| Weight | | | | | | |

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A method for testing pregnant subjects for preeclampsia comprising detecting at least one biomarker associated with preeclampsia in a biological sample from the subject.

2. The method of claim 1, further comprising comparing the abundance of at least one biomarker in the biological sample to a control concentration of at least one biomarker in a control biological sample to identify an increased risk for preeclampsia.

3. The method of claim 2, wherein identifying an increased risk for preeclampsia includes determining that the abundance of the at least one biomarker in the biological sample is detectably higher than the control concentration of at least one biomarker in a control biological sample.

4. The method of claim 2, wherein identifying an increased risk for preeclampsia includes determining that the abundance of the at least one biomarker in the biological sample is detectably lower than the control concentration of at least one biomarker in a control biological sample.

5. The method of claim 1, wherein a preeclamptic subject has at least one biomarker comprising a 718.8 m/z peak and a 0.635±0.85 R_{f} value, a 719.2 m/z and a 0.737±0.072 R_{f} value, a 734.8 m/z peak and a 0.294±0.024 R_{f} value, a 649.3 m/z peak and a 0.343±0.120 R_{f} value, a 507.3 m/z peak and a 0.359±0.039 R_{f} value, 1026.4 m/z peak and a 0.134±0.032 R_{f} value, a 639.3 m/z and a 0.175±0.097 R_{f} value, a 942.5 m/z peak and a 0.915±0.013 R_{f} value, a 1238.5 m/z and a 0.270±0.101 R_{f} value, or any combination thereof.

6. The method of claim 1, wherein a preeclamptic subject has at least two biomarkers comprising a 718.8 m/z peak and a 0.635±0.85 R_{f} value, a 719.2 m/z peak and a 0.737±0.072 R_{f} value, a 734.8 m/z peak and a 0.294±0.024 R_{f} value, 649.3 m/z peak and a 0.343±0.120 R_{f} value, a 507.3 m/z peak and a 0.359±0.039 R_{f} value, 1026.4 m/z peak and a 0.134±0.032 R_{f} value, a 639.3 m/z and a 0.175±0.097 R_{f} value, a 942.5 m/z peak and a 0.915±0.013 R_{f} value, a 1238.5 m/z and a 0.270±0.101 R_{f} value, or any combination thereof.

7. The method of claim 1, wherein a preeclamptic subject has at least three biomarkers comprising a 718.8 m/z peak and a 0.635±0.85 R_{f} value, a 719.2 m/z peak and a 0.737±0.072 R_{f} value, a 734.8 m/z peak and a 0.294±0.024 R_{f} value, 649.3 m/z peak and a 0.343±0.120 R_{f} value, a 507.3 m/z peak and a 0.359±0.039 R_{f} value, 1026.4 m/z peak and a 0.134±0.032 R_{f} value, a 639.3 m/z and a 0.175±0.097 R_{f} value, a 942.5 m/z peak and a 0.915±0.013 R_{f} value, a 1238.5 m/z and a 0.270±0.101 R_{f} value, or any combination thereof.

8. The method of claim 1, wherein a preeclamptic subject has at least four biomarkers comprising a 718.8 m/z peak and a 0.635±0.85 R_{f} value, a 719.2 m/z peak and a 0.737±0.072 R_{f} value, a 734.8 m/z peak and a 0.294±0.024 R_{f} value, 649.3 m/z peak and a 0.343±0.120 R_{f} value, a 507.3 m/z peak and a 0.359±0.039 R_{f} value, 1026.4 m/z peak and a 0.134±0.032 R_{f} value, a 639.3 m/z and a 0.175±0.097 R_{f} value, a 942.5 m/z peak and a 0.915±0.013 R_{f} value, a 1238.5 m/z and a 0.270±0.101 R_{f} value, or any combination thereof.

9. The method of claim 1, wherein the at least one biomarker comprising a 718.8 m/z peak and a 0.635±0.85 R_{f} value is more abundant in a subject at risk for preeclampsia compared to a control.

10. The method of claim 1, wherein the at least one biomarker comprising a 719.2 m/z peak and a 0.737±0.072 R_{f} value is less abundant in a subject at risk for preeclampsia compared to a control.

11. The method of claim 1, further comprising calculating a weighted value derived from biomarker combinations to identify an increased risk for preeclampsia.

12. The method of claim 11, wherein calculating the weighted value comprises [(−5×ratio 734/742)+(33×ratio 1026/518)+(2×ratio 639/582)+(−2×ratio 718/719)]=the weighted value; wherein when the weighted value is less than zero, the subject has an increased risk for preeclampsia, and wherein when the weighted value is greater than zero, the subject is not at risk for preeclampsia.

13. The method of claim 11, wherein calculating the weighted value comprises [(−16×ratio 734/742)+(64×ratio 1026/518)+(3×ratio 639/582)+(1×ratio 1238/623)]=the weighted value; wherein when the weighted value is less than zero, the subject has an increased risk for preeclampsia, and wherein when the weighted value is greater than zero, the subject is not at risk for preeclampsia.

14. The method of claim 1, wherein the one biomarker comprises a peptide, a small molecule comprising a biological amine, a steroid, or other non-peptide biological molecules, or any combination thereof.

15. The method of claim 1, wherein a preeclamptic subject exhibits at least 80% sensitivity.

16. The method of claim 1, wherein the pregnant subject exhibits at least 80% specificity.

17. The method of claim 1, wherein the at least one biomarker is detected in the pregnant subject at 12 to 14 weeks gestation.

18. The method of claim 1, wherein the at least one biomarker is detected at least 3 to 6 months prior to a clinical symptom associated with the preeclampsia.

19. The method of claim 1, wherein the biological sample from the subject comprises serum, plasma, blood, urine, cerebrospinal fluid, amniotic fluid, synovial fluid, cervical fluid, lavage fluid, and combinations thereof.

20. The method of claim 1, wherein the biological sample is serum.

21. The method of claim 1, wherein the biological sample is blood.

22. A biomarker comprising a peptide having a mass ion peak at 718.8 m/z and a 0.635±0.85 R_{f} value; a mass ion peak at 719.2 m/z and a 0.737±0.072 R_{f} value; a mass ion peak at 734.8 m/z and a 0.294±0.024 R_{f} value; a 649.3 m/z peak and a 0.343±0.120 R_{f} value; a 507.3 m/z peak and a 0.359±0.039 R_{f} value; a 1026.4 m/z peak and a 0.134±0.032 R_{f} value; a 639.3 m/z peak and a 0.175±0.097 R_{f} value; a 942.5 m/z peak and a 0.915±0.013 R_{f} value; or a 1238.5 m/z peak and a 0.270±0.101 R_{f} value.

23-30. (canceled)
